(19) United States
(12) Patent Application Publication
(10) Pub. No.: US 2017/0128736 A1
(43) Pub. Date: May 11, 2017

(54) SYSTEM AND METHOD FOR PREVENTING MUSCLE FATIGUE

(71) Applicant: MULTI RADIANCE MEDICAL, Solon, OH (US)

(72) Inventors: Douglas JOHNSON, Brownstown, MI (US); Max KANARSKY, Solon, OH (US)

(21) Appl. No.: 14/936,127

(22) Filed: Nov. 9, 2015

(51) Int. Cl. A61N 2/00; A61N 5/06

(52) U.S. Cl. CPC A61N 2/002; A61N 5/0613; A61N 2/004; A61N 2005/0651; A61N 2005/0659; A61N 2005/0662; A61N 2005/067

(57) ABSTRACT

A method for preventing muscle fatigue induced by exercise in patients with chronic obstructive pulmonary disease is described herein. The method comprises the step of providing a therapeutic laser device, wherein the therapeutic laser device generates a constant magnetic field in combination with a plurality of pulsed lights having a plurality of wavelengths. The method further comprises administering phototherapy to a muscle prior to exercising the muscle by placing the therapeutic laser device in direct contact with skin proximate to the muscle at a plurality of skin sites and pulsing the plurality of lights based on predefined parameters.

### BACKGROUND

Photobiomodulation (“BPM”), also referred to as low level light therapy, is a common technique in which the cells of a human body undergo a chemical reaction upon exposure to light. Low level laser therapy (“LLLT”) in particular, can be used to create therapeutic effects. For example, LLLT can be used to repair damaged tissue, to accelerate recovery from an injury, to help manage pain, and to treat diseases. However, successful and desirable outcomes require proper selection of parameters. For example, altering the strength of light delivered to the body or the amount of time that the light is delivered to the body can significantly influence whether or not desired therapeutic effects are achieved. In some cases, improper selection of parameters can lead to harmful outcomes.

Further, although such therapeutic effects are primarily based on photochemical reactions between the light and the human body cells and not based on thermal changes, existing light therapy devices commonly produce a substantial amount of heat as a byproduct. Light energy will only penetrate to subcuntaneous tissue if it is not completely absorbed by the skin. Photonic energy absorbed by the skin is converted and stored as heat, and the amount of heating is inversely related to the penetration ability of the light. A favorable depth of penetration is related to the selected wavelength and to some degree the mean output power. Excessive amounts of heat, however, may be harmful to the body. Therefore, successful phototherapeutic outcomes require that the light penetrate through the skin, while retaining some absorption properties while limiting the thermal impact on the skin surface.

Moreover, existing light therapy devices commonly utilize a single continuous wavelength light or laser. A continuous wave laser, however, rather than transforming photons into biochemical energy, will convert it to thermal heat, rapidly increasing the skin surface temperature due to a poor penetration profile. This creates a compromise of power versus heat. Since the photochemical and photophysical effects are reduced in response to thermal build up, lower profiles result in greater therapeutic value of the device. But even if aware of this effect, existing devices may be limited in the ability to optimize the balance between the two.

In addition, single wavelength probes may be limited by the specific absorption spectrum of that specific wavelength. This not only determines the depth of penetration, as this is wavelength dependent, but also the biological effect due to the available chromophores for that wavelength, since a photon must be absorbed before any biological process can occur.

Also, LLLT has been limited to creating therapeutic effects in injured or damaged body cells. Various techniques and procedures exist for treating the uninjured to enhance performance. For example, a performance enhancing drug administered prior to an athlete performing may improve the athlete's ability to perform. In another example, consuming a certain diet may enhance an athlete's athletic performance. Various preventative techniques and procedures exist as well. For example, certain diets, medications, or surgeries may be used to improve muscle performance of patients with chronic obstructive pulmonary disease (“COPD”). However, such techniques and procedures may be invasive, inconvenient, illegal, or ineffective.

### SUMMARY

In one example, a method for preventing muscle fatigue induced by exercise in patients with chronic obstructive pulmonary disease is described. The method includes the step of providing a therapeutic laser device, wherein the therapeutic laser device generates a constant magnetic field in combination with a plurality of pulsed lights having a plurality of wavelengths. The method further includes the step of administering phototherapy to a muscle prior to exercising the muscle by placing the therapeutic laser device in direct contact with skin proximate to the muscle at a plurality of skin sites and pulsing the plurality of lights based on predefined parameters.

In one example, a method for reducing muscle fatigue induced by exercise in a patient with chronic obstructive pulmonary disease is described. The method includes the step of generating a constant magnetic field of 35 mT at a skin site proximate to a muscle of a patient, prior to the patient exercising the muscle. The method further includes the step of pulsing a clustered plurality of light sources, including a 905 nm super-pulsed lasers, a 875 nm broadband infrared emitting diode, a 640 nm red light emitting diode, and a 470 nm blue LED, at the skin site concurrent to generating the constant magnetic field.

In one example, a method for reducing muscle fatigue induced by exercise in a patient with chronic obstructive pulmonary disease is described. The method includes the step of generating a constant magnetic field a skin site proximate to a muscle of a patient, prior to the patient exercising the muscle. The method further includes the step of pulsing a clustered plurality of light sources at the skin site for 228 seconds to deliver a 30 J dose of phototherapy treatment concurrent to generating the constant magnetic field.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates example locations on a quadriceps femoris for receiving phototherapy.

FIG. 2A illustrates a comparison of values of isokinetic protocol for PT.

FIG. 2B illustrates a comparison of values of isokinetic protocol for MVIC.

FIG. 3 illustrates the total work with combination of super-pulsed laser and light emitting diodes phototherapy compared to placebo.

FIG. 4 illustrates a comparison of the Borg scale after either phototherapy or placebo.

FIG. 5 illustrates an example thermographic image of an analyzed area and two neighboring areas used as control areas.

FIG. 6 illustrates example doses plotted against temperature in three skin color groups.

FIG. 7 illustrates example doses plotted against temperature in three age groups.

FIG. 8 illustrates example doses plotted against temperatures in male and female groups.

FIG. 9 illustrates an example comparison of 3 different devices.

FIG. 10 illustrates an example comparison of 3 different devices.

FIG. 11 illustrates an example comparison of 3 different devices.

### DETAILED DESCRIPTION

### The Therapeutic Laser Device

Described herein is a therapeutic laser device for providing magnetic laser therapy. The device is designed to maximize the peak outputs of light while also reducing thermal profile. In order to achieve such performance, the device combines multiple wavelengths, light sources, and electromagnetic energy and provides pulsed treatment using irradiation with laser light of low power intensity so that the effects are not due to heat.

The device produces a constant magnetic field and includes a pulsed laser emitter, infrared LEDs, and semiconductor emitters of visible light. Emitters are arranged in groups containing a semiconductor emitter of visible light, infrared light guide and/or pulsed emitter. The device is equipped with elements of radiation summation for each group of emitters made from optical material in the form of a light guide.

In one example, the therapeutic laser device includes between one and four 905 nm super-pulsed lasers, four 875 nm broadband infrared emitting diodes, between two and four 640 nm red light emitting diodes, between zero and two 470 nm blue LEDs, and a static magnetic field of 35 mT. This combination of wavelengths and light sources clustered together into a single probe, optimizes the biological effects of the phototherapeutic window, provides a greater depth of penetration, and eliminates the thermal barrier. The device operates with two or more wavelengths and light sources operating concurrently in pulsed and super pulsed modes.

The pulsing operating modes results in minimized heat. In particular, even though the therapeutic laser device creates a desired higher peak power, there is little resulting heat accumulation within a target tissue due to the ultrashort pulses. The combination of wavelengths in the device helps to improve the percentage of available light at greater tissue depths. This resolves any issues with the inefficient use of higher-powered outputs in continuous wave devices and a poor penetration profile.

The combination of and concurrent use of different wavelengths also allows for more efficient triggering of the phototherapeutic response. In particular, concurrent use of different wavelengths provides an overlapping effect of peak activation that accelerates cytochrome c oxidase (“CCO”) activity. Rather than attempting to increase the activity of CCO with a single wavelength with a higher MOP and just one set peak time profile, the use of additional wavelengths with lower dose and different time profiles increases the total time of peak activation. This allows for less power to be used across all wavelengths, rather than using a single higher output one.

Each wavelength and light source must create a synergistic effect, that when combined with others, summates greater than the individual effects. The combination of a super-pulsed laser, infrared LEDs, and red LEDs optimizes the entire CCO peak activation profile for enhancing ATP production, stimulating NO release and activating ROS. The concurrent multiple wavelengths span the entire therapeutic light spectrum to reach varying depths of penetration while creating non-thermal synergy that improves overall penetration. This, in turn, creates a favorable mix of the available parameters to maximize therapeutic outcomes in the clinic for consistent and reliable results.

To maximize the therapeutic outcomes, the parameters may be adjusted as appropriate. For example, the number of times a laser fires per seconds is the “frequency” of the pulses and affects the mean output of power that the laser delivers. This, in turn, impacts the amount of light that the tissue receives. By changing the frequency, the rate of energy delivered is also changed. Based upon tissue response or need, the dose can be delivered in a shorter amount of time by increasing the frequency output of the therapeutic laser device or spread out over a longer time period by lowering the rate of the laser firing. In essence, it works like a thermostat. This allows for the therapeutic laser device to deliver energy in a more customizable manner.

In addition to the super-pulsed lasers, a therapeutic laser device contains several infrared emitting diodes (IREDs) and light emitting diodes (LEDs). The addition of visible and infrared wavelengths and light sources provided by both spectrums allows broader coverage of the therapeutic spectrum. Both IREDs and LEDs will, if left on continuously, exhibit the same thermal profile as a continuous wave laser. That is, the increase in power output would also increase the beating effect, due to the inefficiency of the semiconductor processes that generate light. To work in concert with the super pulsing laser, both IREDs and LEDs are also pulsed to reduce photothermal effects on tissue.

In one example, the device may be controlled by a desktop unit via AC power. In one example, the device may be portable and cordless and include an onboard rechargeable battery. In one example, the device may be a wearable device. For example, the therapeutic laser device may be incorporated into a watch, a wristband, or some sort of strap around the leg or other body part that needs therapy. The device may be programmed to automatically deliver light treatment to the desired area at defined times and intervals, for example.

Preventing Muscle Fatigue

Using appropriate parameters, the therapeutic laser device can be used to treat non-injured persons to prevent disease or injury from occurring. For example, the therapeutic laser device can provide a non-pharmacological and non-invasive means for preventing muscle fatigue and therefore improving performance and also reduce risk of injury. This can be applicable to athletes who seek to increase performance and post exercise recovery time but also for COPD patients seeking to prevent muscular fatigue induced by exercise.

To establish the effects of the therapeutic laser device on preventing muscle fatigue in patients with COPD and to establish the optimal parameters for such an application, the following study was performed.

Methods

Thirteen patients were consecutively recruited from the outpatient chronic pulmonary diseases clinic at the Nove de Julho University. All patients had a diagnosis of COPD according to the global initiative for chronic obstructive lung disease. The patients were at a stable phase of the disease indicated by no change in the medical therapy (including oral steroids) or exacerbation of symptoms in the preceding 4 weeks. Patients with other known severe chronic diseases, including cardiac, neuromuscular, or orthopedic disorders, were excluded. The study was approved by the institutional ethics committee, and written informed consent was obtained from all patients.

Randomization was performed by simple drawing of lots, which was used to determine whether the active combination of super-pulsed laser and LEDs phototherapy or placebo would be given at the first session. Participants were crossed over to receive whichever treatment was not given at the first session. Randomization labels were created by using a randomization table at a central office where a series of sealed, opaque, and numbered envelopes were used to ensure confidentiality. A participating researcher who had the function of programming the phototherapy device based on the randomization results conducted randomization. This researcher was instructed not to inform the participants or other researchers regarding the phototherapy dose. Thus, the researcher in charge of the administration of the phototherapy was blinded to the dose applied to the volunteers. Blinding was further maintained by the use of opaque goggles by the participants.

Procedures

A crossover, double-blinded, placebo-controlled, and randomized clinical trial was carried out. The study was conducted in the Laboratory of Phototherapy in Sport and Exercise at the Nove de Julho University, São Paulo, Brazil. Patients were administered either phototherapy or placebo treatments on two visits, 1 week apart. Immediately after the application, the maximum voluntary isometric contraction (MVIC) was determined and the endurance test—total work (TW).

Spirometry was performed as per the American Thoracic Society/European Respiratory Society criteria; FVC, FEV1, and FEV1/FVC are expressed as absolute values and percent of predicted.

An isokinetic dynamometer was used for the evaluation of muscle function and the execution of the exercise protocol. For the MVIC test, the volunteers sat at an angle of 100° between the trunk and hips with the non-dominant leg positioned with the knee at 60° of flexion (0° corresponds to complete knee extension) and were strapped to the dynamometer seat. The dominant leg was positioned at 100° of hip flexion and was strapped to the seat. The volunteers were fastened to the seat of the dynamometer by using two additional straps crossing the trunk. The volunteers were instructed to cross their arms over their trunk, and the axis of the dynamometer was positioned parallel to the center of the knee.

The MVIC test consisted of three 5-s isometric contractions of the knee extensors of the non-dominant leg. The highest torque value of the three contractions (peak torque [PT]) was used for the statistical analysis. This parameter was chosen because it reflects the maximum generation of force by the muscle. Instructions on how to execute the test were given prior to testing, and the volunteers received verbal encouragement during the execution of the test.

A resting period of 60 s was allowed, following the MVIC test after which volunteers performed a familiarization isokinetic protocol. The familiarization consisted of five submaximal voluntary repetitions of knee flexion-extension in an eccentric contraction protocol, followed by a resting period of 60 s. The eccentric contraction protocol consisted of 20 eccentric, isokinetic contractions of the knee extensor musculature in the non-dominant leg (two sets of ten repetitions, 30 s rest intervals between sets) at a velocity of 60° seg-1 in both the eccentric and concentric movements with a 60° range of motion (between 90° and 30° of knee flexion). At each contraction, the dynamometer automatically (passively) positioned the knee at 30°; the dynamometer then flexed the knee until reaching 90°. The volunteers were instructed to resist against knee flexion movement imposed by the dynamometer with maximum force. The researcher in charge of the eccentric contractions protocol was blinded to randomization and allocation of volunteers to experimental groups.

Before and after the endurance test, the perceived effort (dyspnea and leg fatigue) was assessed by using the modified Borg scale.

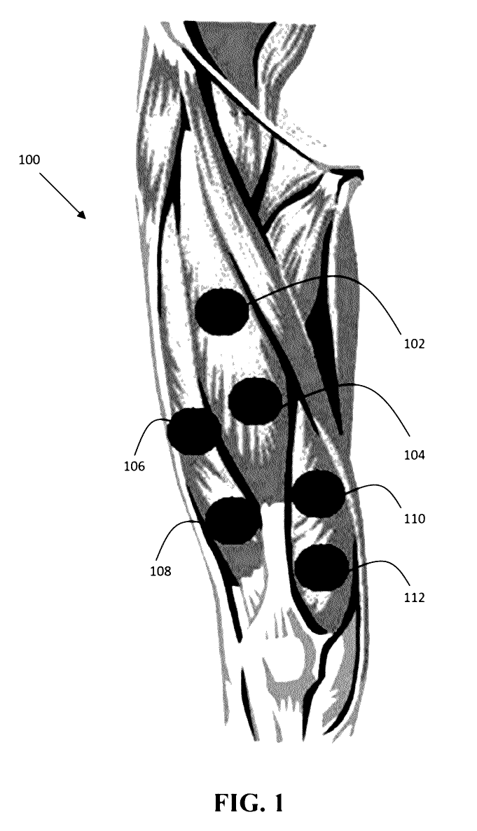

Patients received a single application of either combined super-pulsed laser and LED phototherapy or placebo 1 week apart. The phototherapy combining super-pulsed laser and LEDs or placebo was administered using the above described therapeutic laser device immediately before the testing of lower limb isokinetic dynamometry. In view of the extensive area of radiation employed in this project, the use of clusters is fundamental to the application of the therapy. The application of phototherapy was held with the cluster in direct contact with the skin, at six sites of the quadriceps femoris, as illustrated in FIG. 1. In particular, the therapeutic laser device was held in direct contact at two central sites at the vastus intermedius muscle 102 and 104, at two lateral sites at the vastus lateralis muscle 106 and 108, and at two medial sites at the vastus medialis muscle 110 and 112. For placebo, the same procedures were performed, but without irradiation. During the application of combined super-pulsed laser and LEDs phototherapy or placebo, the patient wore protective goggles to prevent them from seeing whether or not there was light being radiated.

Since the cluster has 12 diodes that were used to irradiate six different locations of the extensor muscles of the knee 100 as illustrated in FIG. 1, a total of 72 points in the musculature were irradiated. Phototherapy parameters were chosen based on research performed to identify, optimal parameters for such an application of laser treatment that would achieve desired results. In particular, research was performed by employing three different doses of 10 J, 30 J and 50 J before it was determined that a 30 J dose was the optimal dose to achieve the desired results. Table 1 provides a full description of the phototherapy parameters.

TABLE 1
Parameters

| Number of lasers | 4 super-pulsed infrared |
|---|---|
| Wavelength (nm) | 905 |
| Frequency (Hz) | 250 |
| Peak power (W)-each | 12.5 |
| Average optical output (mW)-each | 0.03125 |
| Power density (mW/cm²)-each | 0.07 |
| Dose (J)-each | 0.07125 |
| Spot size of laser (cm²)-each | 0.44 |
| Number of red LEDs | 4 red |
| Wavelength of red LEDs (nm) | 640 |
| Frequency (Hz) | 2 |
| Average optical output (mW)-each | 15 |
| Power density (mW/cm²)-each | 16.66 |
| Dose (J)-each | 3.42 |
| Spot size of red LED (cm²)-each | 0.9 |
| Number of infrared LEDs | 4 infrared |
| Wavelength of infrared LEDs (nm) | 875 |
| Frequency (Hz) | 16 |
| Average optical output (mW)-each | 17.5 |
| Power density (mW/cm²)-each | 19.44 |
| Dose (J)-each | 3.99 |
| Spot size of LED (cm²)-each | 0.9 |
| Magnetic field (mT) | 35 |
| Irradiation time per site (sec) | 228 |
| Total dose per site (J) | 30 |
| Total dose applied in muscular group (J) | 180 |
| Aperture of device (cm²) | 20 |

Using the identified parameters, phototherapy was performed by delivering 30 J per site for 228 seconds, or 180 J of total irradiated energy on muscle for a total irradiation time of 1,368 seconds.

The intention-to-treat analysis was followed. The Kolmogorov-Smimov test was used to verify the normal distribution of data. Parametric data were expressed as mean and standard deviation. Non-parametric data were expressed as median and interquartile intervals. Differences in the variables of muscle function between combined phototherapy and placebo treatments were compared by using paired, two-sided Student's t-tests, and the differences of Borg scale were compared by using the Wilcoxon test. The level of statistical significance was set at p<0.05.

Results

The volunteer population was formed mostly by patients with moderate COPD according to the GOLD criteria (GOLD 2, n=7), with the remaining patients classified as having mild (GOLD 1, n=1), severe (GOLD 3, n=4), and very severe (GOLD 4, n=) COPD. Table 2 summarizes the characteristics of the patients.

TABLE 2
Characteristics

| Variables | Mean ± SD |
|---|---|
| Age, years | 61 ± 6 |
| BMI, kg/m² | 24.3 ± 4.1 |
| FVC, L (% predicted) | 2.5 ± 0.7 (74 ± 15) |
| FEV₁, L (% predicted) | 1.2 ± 0.4 (53 ± 16) |
| FEV₁/FVC ratio, % | 60.3 ± 12.2 |

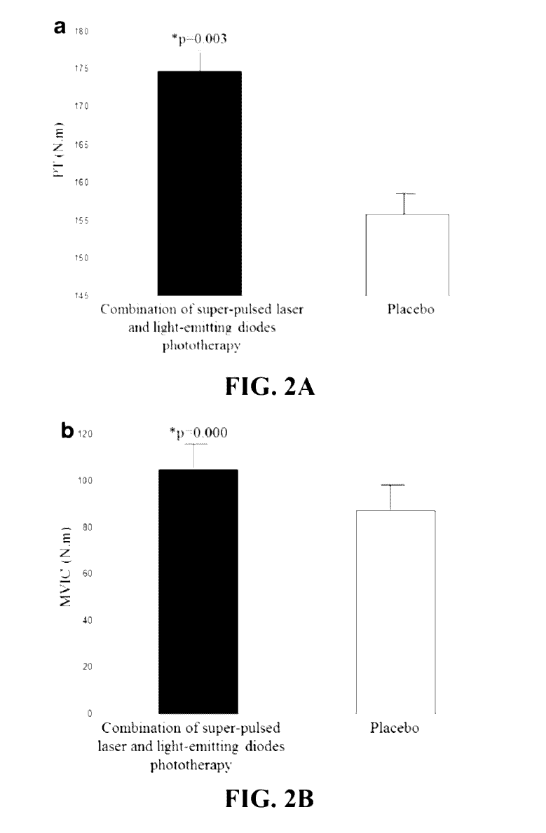

A statistically significant difference was found for the increase of PT after the application of combined superpulsed laser and LED phototherapy when compared with the placebo (174.7±35.7 N·m vs. 155.8±23.3 N·m, respectively; p=0.003), as illustrated in FIG. 2A. A similar finding was found for MVIC, with values of 104.8±26.0 N·m vs. 87.2±24.0 N·m for the phototherapy treatment and placebo, respectively (p=0.000), as illustrated in FIG. 2B.

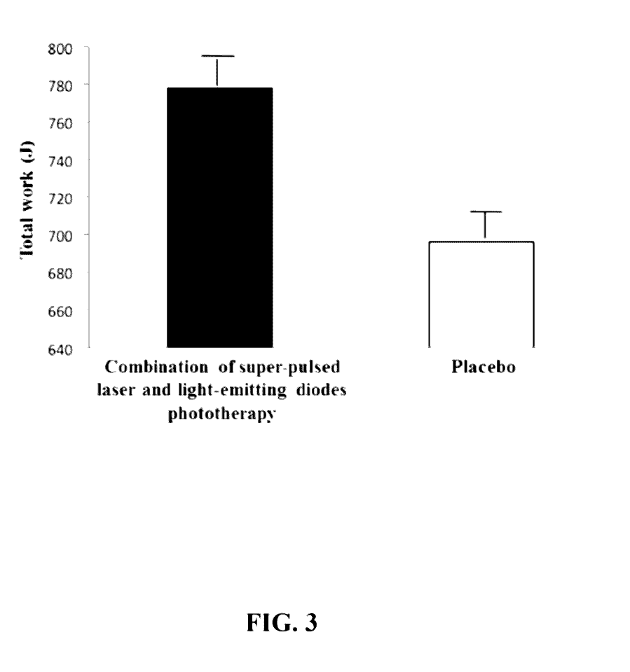

As illustrated in FIG. 3, a greater value in the TW was observed during endurance testing with the combination of super-pulsed laser and LED phototherapy when compared to the placebo (778.0±221.1 J vs. 696.3±146.8 J, respectively; p=0.005).

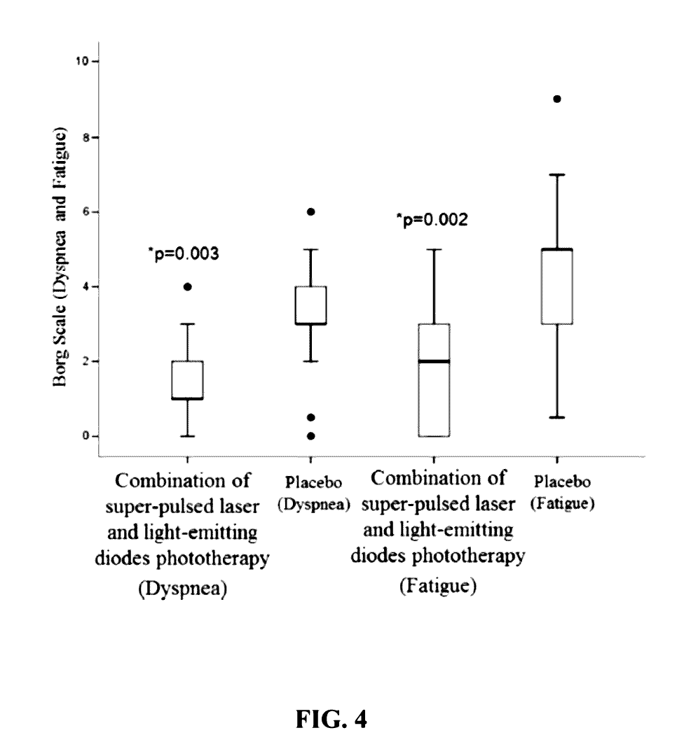

As illustrated in FIG. 4, the dyspnea score after the combination of super-pulsed laser and LED phototherapy was lower in comparison with the placebo (1 [0-4] vs. 3 [0-6], p=0.003). A similar result was seen in the fatigue score for the lower limbs (2 [0-5] vs. 5 [0.5-9], respectively; p=0.002).

In summary, it was demonstrated through the study described that a combination of super-pulsed laser and LED phototherapy on the femoral quadriceps muscle in patients with COPD was able to increase PT by 20.2% and TW by 12%. Furthermore, combined phototherapy prior to exercise led to a decreased sensation of dyspnea and lower limb fatigue in patients with COPD.

It should be appreciated that, although the study describes the treatment of extensor muscles of the knee, similar techniques can be used to treat other suitable muscle groups in different parts of the body.

It should be further appreciated that the identified parameters may be suitable for treating bodies having various ages and skin pigmentations without concern of damaging the skin as a result of thermal effects.

Thermal Impact of Phototherapy

The following study was performed to evaluate the thermal influence during phototherapy of concurrent multiple wavelengths and light sources on human skin and to confirm that the identified parameters are safe and effective for humans of varying age and skin pigmentation.

Subjects

A sample of 42 healthy adult volunteers, male and female, greater than 18 years of age, was recruited. Subjects were separated by gender and age and stratified according to skin color using Von Luschan's chromatic scale, which ranks color from 1 (lightest skin) to 36 (darkest skin). Three categories were created to rank skin pigmentation: 1 to 15 corresponding to light skin, 16 to 28 corresponding to medium skin, and 29 to 36 corresponding to dark skin. Participants were additionally stratified according to age (under 40 years of age, between 40 and 60 years of age, and over 60 years of age) due to changes in skin optical properties during aging. Any individual with a history of skin disease was excluded from the study.

Instruments

All skin temperature readings were measured by a thermographic camera (Flir System, ThermaCAM T400). The ancillary software (ThermaCAM Researcher Pro 2.8 SR-1) included tools to quantify the recorded temperatures. The temperatures were measured with a precision of 50 mK at 30° C. with an accuracy of ±2% (product information). The example therapeutic laser device and the above described doses parameters was used to deliver the light treatment to the subjects.

Experimental Procedure

To acclimate skin temperatures to the surroundings, patients were instructed to remain in the laboratory for 15 min prior to the start of the experiment and remain seated during the entire experiment. All participants were instructed to report to the investigator any sensation of heat felt during the test on the irradiated area and report if the heat became painful or uncomfortable and the intervention needed to cease.

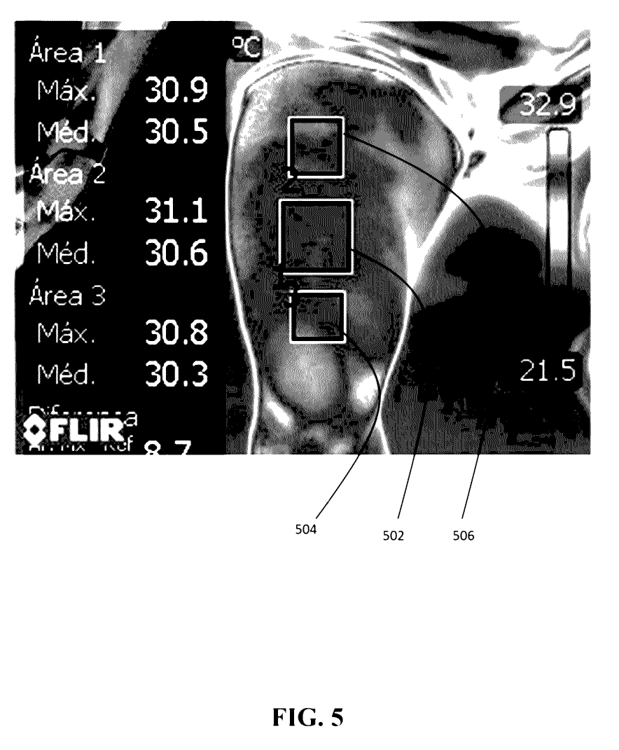

The anterior 502, central aspect of the non-dominant thigh was selected as the target for the irradiation. Two adjacent areas 504 and 506 of the same anterior thigh (proximal and distal to the treatment area) were used as controls, as illustrated in FIG. 5. Two investigators performed the study, one operated the thermographic camera and the second administered the therapy. Each trial lasted approximately 30 min and five skin temperature measurements were recorded for each volunteer. A baseline measurement was taken prior to the first irradiation followed by four additional measurements during the treatment that corresponded to the predetermined doses based upon time (placebo, 60 s; 10 J, 76 s; 30 J, 228 s; and 50 J, 381 s) with a 3-min pause observed between doses. The sequence of the doses (and times) was the same for all volunteers. The placebo comparator was delivered using the same laser device as the active interventions but was powered off and the participants were unaware. The laser emitter was held stationary approximately 10° degrees from vertical and in direct contact with the skin of the anterior central aspect of the non-dominant thigh.

All study participants, as well as the operator of the thermographic camera, were blinded to the assignment of the active and placebo comparators. Thermography readings were recorded during the final 5 s of each irradiation dose and continued for 1 min following the conclusion of the irradiation (a total of 1 min and 5 s). The maximum temperatures from the irradiated area and two control areas (proximal and distal) were simultaneously registered by ThermaCAM.

Results

Forty two volunteers with mean age of 50.60 years old (±19.82), mean weight of 76.45 kg (±18.92), and mean height of 169.00 cm (±10.00) were recruited to participate in the study with sub-categorization of 14 volunteers with dark skin, 14 with medium skin, and 14 with light skin. Fourteen volunteers were under 40 years old, 14 were between 41 to 60 years old, and 14 were over 60 years old. Furthermore, 21 volunteers were male and 21 were female. Distribution of volunteers among groups is summarized in Table 3.

TABLE 3
Volunteers

| | | | Light | Medium | Dark | |
|---|---|---|---|---|---|---|
| <40 | years | 7 male + 7 female = 14 | 2 (1 m + 1f) | 6 (3 m + 3f) | 6 (3 m + 3f) | Total: 14 |
| 41-60 | years | 7 male + 7 female = 14 | 6 (3 m + 3f) | 4 (2 m + 2f) | 4 (2 m + 2f) | Total: 14 |
| >61 | years | 7 male + 7 female = 14 | 6 (3 m + 3f) | 4 (2 m + 2f) | 4 (2 m + 2f) | Total: 14 |
| | | Total: 21 male and 21 female | Total: 14 | Total: 14 | Total: 14 | |

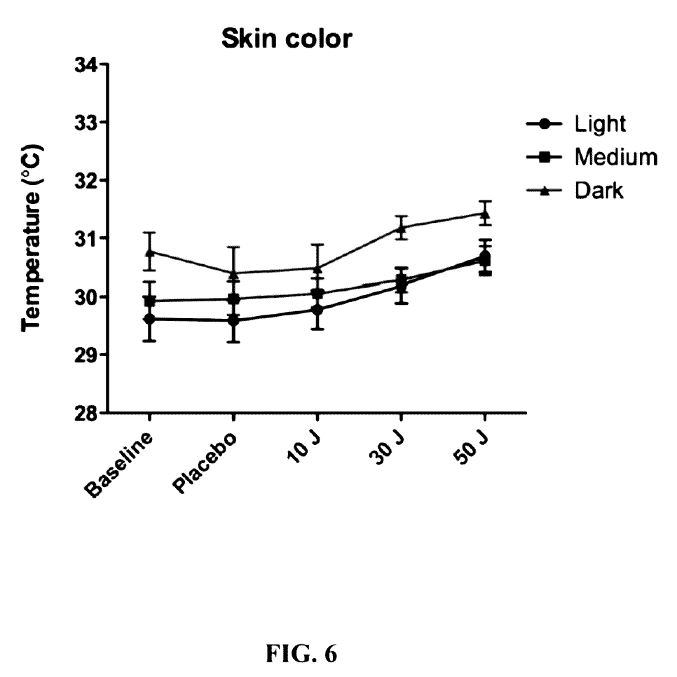

As illustrated in FIG. 6, other than a slight, non-significant increase (p<0.05), no differences from baselines were observed between light, medium, or dark skin pigmentation.

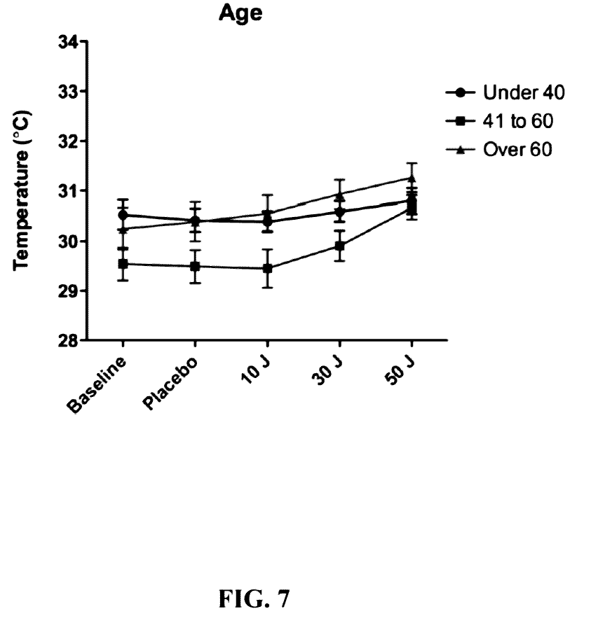

As illustrated in FIG. 7, no significant difference between the three age groups can be observed from baseline either. The concurrent use of super-pulsed laser, and red and infrared LEDs did produce a small, non-significant (p<0.05) increase in skin temperature when the larger doses were applied.

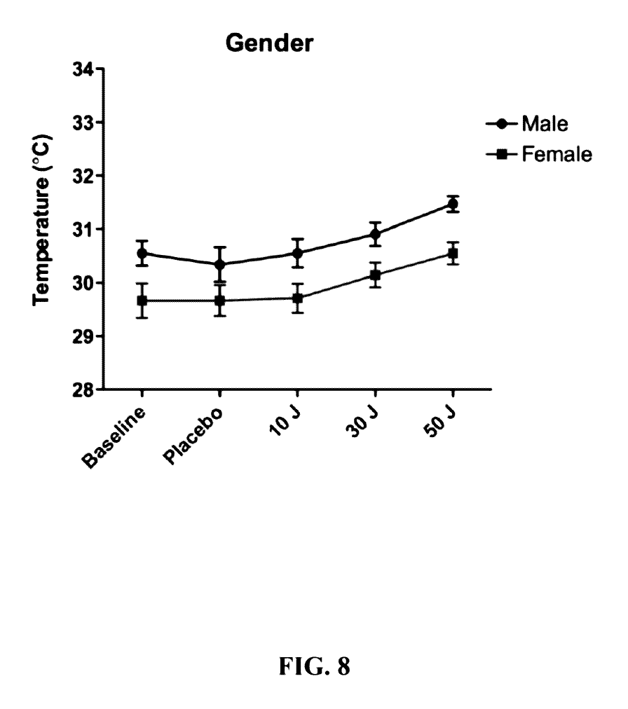

Finally, as illustrated in FIG. 8, other than a slight, non-significant increase (p<0.05), no differences from baselines were observed between age groups in regard to gender.

Thus, the concurrent use and combination of super-pulsed lasers, and red and infrared LEDs is safe and can be regardless of degree of skin pigmentation without concern of damaging thermal effects to the skin.

It should further be appreciated that, although alternative devices for delivering light therapy, other than the therapeutic laser device described herein, may be commercially available, the alternative devices may not produce desirable outcomes achieved by the class 1 laser therapeutic device, including the super-pulsed lasers and LEDs, described herein. The following study was performed to evaluate the observed effects on skeletal muscle performance and post-exercise recovery by three different, readily available phototherapy devices to establish a clear understanding of the parameters necessary for optimal use of phototherapy in muscle performance and recovery.

Comparison Between Three Devices

Materials and Methods

Forty healthy untrained male subjects were recruited and participated in a study to evaluate the effects of phototherapy on skeletal muscle performance and post-exercise recovery with three devices to determine how the ergogenic and protective effects on skeletal muscle tissue are affected by different device parameters. The devices included a Class 4 device (manufactured by LiteCure—USA), a Class 3B device (manufactured by Thor—UK) and a Class 1M therapeutic laser device described herein.

The inclusion criteria included male subjects, between 18 and 35 years old that perform less than two sessions of exercise weekly with either light or intermediate skin color. Any volunteer who presented with a preexisting musculoskeletal injury to hips or knees in the previous two months, utilizes any pharmacological agents or nutritional supplements regularly, or was injured during the study was to be excluded from the participation. The volunteers were randomly allocated to four experimental groups (n=10 per group) according to the phototherapy dose. Randomization was carried out by a simple drawing of lots.

For placebo treatments, all three devices were employed. Four volunteers were treated with placebo mode of device A, three with device B, and three with device C. Randomization labels were created using a randomization table at a central office where a series of sealed, opaque, and numbered envelopes were used to ensure confidentiality. A participating researcher who had the function of programming each phototherapy device based on the randomization results was instructed to not inform the participants or other researchers regarding the settings. Thus, the researcher in charge of the administration of the phototherapy was blinded to the dose applied to the volunteers.

Experimental Protocol

Blood samples (10 ml) were taken from the antecubital vein of each volunteer before and one minute after the eccentric contraction protocol by a qualified nurse blinded to the allocation of the volunteers in the four experimental groups. One hour following collection each sample was centrifuged at 3000 rpm for 20 minutes. Pipettes were used to transfer the serum to Eppendorf® tubes, which were stored at −80° C. until analysis. Additional blood samples were collected 1, 24, 48, 72 and 96 hours after the exercise protocol.

CK activity was determined using spectrophotometry and specific reagent kits (Labtest®, São Paulo—SP, Brazil). The CK activity was performed by a blinded researcher.

A visual analogue scale (VAS) of 100 mm evaluated DOMS used as a self-rating of volunteers DOMS intensity, with assistance of a blinded researcher.

Prior to the isokinetic protocol, each volunteer actively stretched the non-dominant knee extensors three times for sixty seconds each. Stationary bike pedaling set at 100 RPM and without load for five minutes each was used as a general warm up activity.

Following warm-up, MVC tests were performed with the isokinetic dynamometer (System 4, Biodex®, USA) to assess muscle function. Each volunteer was fixated to the dynamometer at an angle of 100° between the trunk and hip and instructed to cross their arms. The non-dominant leg was positioned at 60° of knee flexion (0° corresponds to complete knee extension) and the dominant at 100° of hip flexion.

The MVC test consisted of three five-second isometric contractions of the knee extensors of the non-dominant leg. The highest peak torque was used for the statistical analysis. The MVC was performed also immediately (1 minute) after the eccentric contraction protocol as well as 1, 24, 48, 72 and 96 hours after. The researcher performing the assessment of MVC was blinded to randomization and allocation.

Phototherapy

The devices used to perform phototherapy included a high powered continuous wave Class 4 device (manufactured by LiteCure—USA), a continuous wave low-level Class 3B device (manufactured by Thor—UK) and a Class 1M therapeutic laser device described herein containing a combination of super-pulsed lasers and red and infrared LEDs.

An 180 J dose and parameters for Class 1M and 3B lasers were selected as previously described. Both LLLT devices were applied in direct contact with the skin at six sites of the quadriceps femoris, also as previously described. While the same dose was applied also to the Class 4 group, the application was done with contact in a scanning method. This was per manufacturer's specific instructions, in order to avoid any potential damaging thermal effects.

To ensure blinding, all devices emitted the same sounds regardless of the programmed mode/dose and opaque goggles worn by volunteers to provide safety and to keep the double blind condition. Optical power was calibrated before irradiation in each volunteer using a Thorlabs thermal power meter. The researcher that performed phototherapy was blinded to randomization and allocation of volunteers.

Following treatment, volunteers performed the eccentric contraction protocol of 75 eccentric isokinetic contractions of the knee extensor of the non-dominant leg (5 sets of 15 repetitions, 30-second rest interval between sets) at a velocity of 60°.seg⁻¹ in both the eccentric and concentric movements with a 600 range of motion (between 90° and 30° of knee flexion). At each contraction, the dynamometer automatically positioned the knee at 30°. The dynamometer then flexed the knee until reaching 90°. The volunteers were instructed to resist against knee flexion movement imposed by the dynamometer with maximum force. The researcher performing the protocol was blinded to randomization and allocation of volunteers.

Results

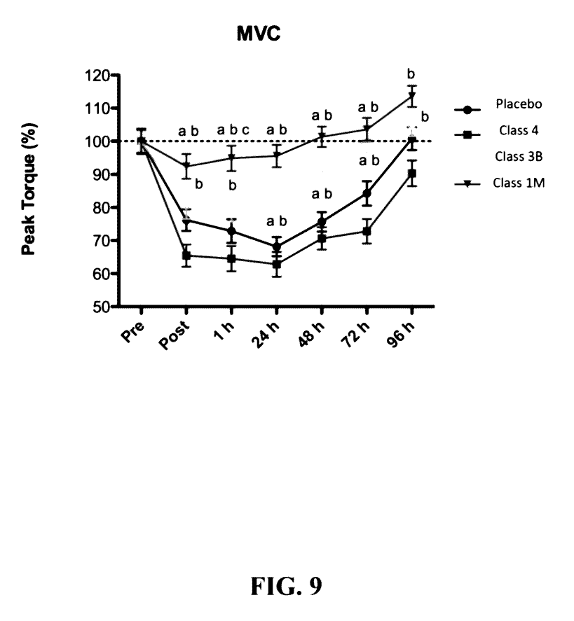

As illustrated in FIG. 9, only the Class 1M group was able to maintain the MVC compared to placebo (p<0.05) and performed significantly better than the Class 4 group (p<0.05) immediately after the active treatment up to 96 hours later. Also, there was a noted increase in MVC at 48, 72 and 96 hour time points. The Class 3B group was also significantly better than placebo (p<0.05) but only in the time frame between 24 to 72 hours after eccentric exercise and statistically better than the Class 4 group (p<0.05) at all time points. It suggests that the two low level devices out performed (p<0.05) both the high powered laser device and the placebo.

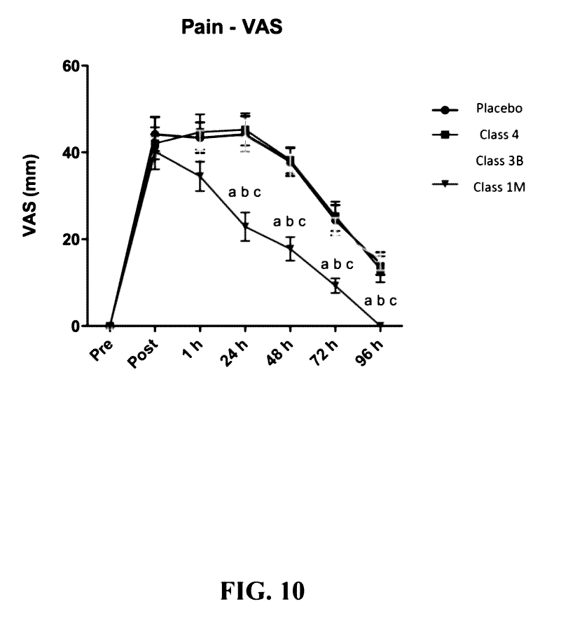

Regarding DOMS measured by VAS, only the Class 1M device was able to effectively reduce pain compared to the placebo, Class 3B and 4 devices (p<0.05) beginning at the 24 hour time point until the end of the data collection at 96 hours, as illustrated in FIG. 10.

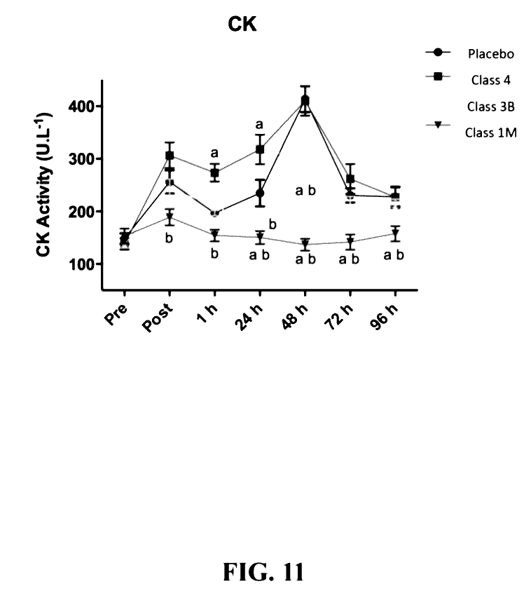

As illustrated in FIG. 11, CK analysis reveals that the Class 1M group was able to prevent the exercise induce increase in CK activity starting at 24 hours until the 96 hours after exercise (p<0.05) compared to placebo and to the Class 4 device in all experimental times (p<0.05). The Class 3B device was significantly able to decrease CK activity compared to the placebo (p<0.05), at a single time point of 48 hours post exercise and also when compared to the Class 4 group (p<0.05) at 24 and 48 hours.

Also, the Class 4 group did not demonstrate a positive effect (p>0.05) on CK activity compared to any of the experimental groups. In fact, the Class 4 group had a statistically significant increase in CK activity (p<0.05) when compared to that of the placebo group at 1 and 24 hours.

Thus, the Class 1M device demonstrated superior and more consistent results than either the Class 3B or 4 devices in all outcome measures when compared to placebo.

It should be appreciated that, although the examples described herein refer to preventing muscle fatigue, the example therapeutic laser device using the example parameters described may similarly be used for other preventative purposes. For example, the therapeutic laser device using the example parameters described may be used to prevent onset of diseases such as Duchenne Muscular Dystrophy.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

What is claimed:

1. A method for preventing muscle fatigue induced by exercise in a patient with chronic obstructive pulmonary disease, comprising the steps of:
providing a therapeutic laser device, wherein the therapeutic laser device generates a constant magnetic field in combination with a plurality of pulsed lights having a plurality of wavelengths; and
administering phototherapy to a muscle prior to exercising the muscle by placing the therapeutic laser device in direct contact with skin proximate to the muscle at a plurality of skin sites and pulsing the plurality of lights based on predefined parameters.

2. The method of claim 1, wherein the therapeutic laser device comprises a pulsed laser emitter, an infrared LED, and a semiconductor emitter of visible light.

3. The method of claim 2, wherein the pulsed laser emitter, an infrared LED, and a semiconductor emitter of visible light are clustered into a single probe.

4. The method of claim 2, wherein the therapeutic laser device comprises four pulsed laser emitters, four infrared LEDs, and four semiconductor emitters of visible light clustered into a single probe.

5. The method of claim 4, wherein placing the therapeutic laser device in direct contact with skin proximate to the muscle at a plurality of skin sites comprises placing the therapeutic laser device in direct contact with skin proximate to the muscle at six skin sites, wherein seventy two total skin points are irradiated.

6. The method of claim 1, wherein the predefined parameters comprise a 30 j dose per skin site.

7. The method of claim 6, wherein placing the therapeutic laser device in direct contact with skin proximate to the muscle at a plurality of skin sites comprises placing the therapeutic laser device in direct contact with skin proximate to the muscle at six skin sites, and wherein the predefined parameters comprise an 180 j total dose for the muscle.

8. The method of claim 1, wherein the predefined parameters comprise phototherapy being delivered to a site for a total time of 228 seconds.

9. The method of claim 1, wherein the predefined parameters comprise a pulsed laser wavelength of 905 nm.

10. The method of claim 1, wherein the predefined parameters comprise an infrared LED wavelength of 875 nm, a semiconductor emitter of visible light wavelength of 640 nm, and a magnetic field of 35 mT.

11. The method of claim 1, wherein the therapeutic laser device is placed in direct contact with one of light skin, medium skin, and dark skin.

12. The method of claim 1, wherein the therapeutic laser device comprises a Class 1 device.

13. The method of claim 1, wherein the therapeutic laser device comprises a super-pulsed laser emitter.

14. The method of claim 1, wherein the therapeutic laser device is portable, comprising a rechargeable battery.

15. The method of claim 1, wherein the therapeutic laser device is wearable, and wherein providing the therapeutic laser device comprises securing the therapeutic laser device to a portion of a human body proximate to the muscle.

16. The method of claim 15, wherein the wearable device is pre-programmed to automatically administer phototherapy to the muscle according to predefined times and intervals.

17. A method for reducing muscle fatigue induced by exercise in a patient with chronic obstructive pulmonary disease, comprising the steps of:
generating a constant magnetic field of 35 mT at a skin site proximate to a muscle of a patient, prior to the patient exercising the muscle; and
pulsing a clustered plurality of light sources, including a 905 nm super-pulsed lasers, a 875 nm broadband infrared emitting diode, a 640 nm red light emitting diode, and a 470 nm blue LED, at the skin site concurrent to generating the constant magnetic field.

18. The method of claim 17, wherein pulsing the clustered plurality of light sources comprises delivering a 30 j dose to the skin site.

19. The method of claim 17, wherein pulsing the clustered plurality of light sources comprises pulsing the plurality of light sources at the skin site for a total time of 228 seconds.

20. A method for reducing muscle fatigue induced by exercise in a patient with chronic obstructive pulmonary disease, comprising the steps of:
generating a constant magnetic field a skin site proximate to a muscle of a patient, prior to the patient exercising the muscle; and
pulsing a clustered plurality of light sources at the skin site for 228 seconds to deliver a 30 J dose of phototherapy treatment concurrent to generating the constant magnetic field.
